(19) United States
(12) Patent Application Publication
(10) Pub. No.: US 2009/0186405 A1
(43) Pub. Date: Jul. 23, 2009

(54) Rapid Chilling Device for Vitrification

(76) Inventor: Milton Chin, Trumbull, CT (US)

Correspondence Address: MARK S. NOWOTARSKI, 30 GLEN TERRACE, STAMFORD, CT, 06906, US

(21) Appl. No.: 12/355,027

(22) Filed: Jan. 16, 2009

Related U.S. Application Data
(60) Provisional application No. 61/021,661, filed on Jan. 17, 2008.

(51) Int. Cl. C12M 1/00; F17C 13/00

(52) U.S. Cl. 435/307.1; 62/45.1

(57) ABSTRACT

Successful cryopreservation by the vitrification method depends on high chilling speed. Practitioners of vitrification prefer to use liquid nitrogen as the chilling cryogen due to its inherent safety and low cost. Plunging vitrification cryocontainers in to a quiescent pool of liquid nitrogen invariably results in a chilling rate less than the theoretical potential. The shortfall is attributed to the well-known Leidenfrost effect. The purpose of this invention it to provide improve chilling rates during vitrification using liquid nitrogen. One feature of this invention is a contacting device that invokes convective heat transfer principles to increase chilling speed. In another feature of this invention, cryogen velocity is derived from a self-pressurized dewar containing a saturated cryogen. The self-pressurization is achieved by ambient heating of the dewar's contents. In another embodiment, a sub-cooled cryogen, such as propane, is used in tandem with a saturated cryogen, such as LN2, in a self-pressurized dewar.

### CROSS-REFERENCE TO RELATED APPLICATIONS

This application claims priority to U.S. provisional patent application entitled “Rapid Chilling Device for Vitrification”, Ser. No. 61/021,661 filed on Jan. 17, 2008. Said provisional application is incorporated herein by reference.

### TECHNICAL FIELD

This invention is in the field of devices for the cryopreservation of biological specimens.

### BACKGROUND

Cryopreservation is practiced in the life sciences for the purpose of halting biological activity in valuable cell(s) for an extended period of time. One factor in the success of cryopreservation is reducing or eliminating the deleterious effect of ice crystal formation. Sophisticated methods are needed to thwart the natural tendency of water to freeze into ice during cryopreservation. “Vitrification” is such a method. Vitrification can be described as a rapid increase in fluid viscosity upon fast chilling that traps water molecules in a random orientation. Its success is predicated on avoiding the formation of cell-damaging ice altogether.

### Vitrification

The initial step in vitrification is to dehydrate a cell or cells with an aqueous solution (“vitrification media”) containing permeating and/or non-permeating cryoprotectants (“CPA”). The cell or cells, together with a small quantity of vitrification media, comprise a “biological specimen.” The biological specimen is then placed in a suitable cryocontainer. A cryocontainer is a container that is suitable for use at cryogenic temperatures. As used herein, “cryogenic temperatures” means temperatures colder than −80° C.

The biological specimen is then rapidly chilled by immersion in a cryogenic fluid, such as liquid nitrogen (“LN2”). With a proper combination of chilling speed and CPA concentration, intracellular water will attain a solid, innocuous, glassy (vitreous) state rather than an orderly, damaging, crystalline ice state.

Clinical vitrification has two primary goals. The first is long term storage in a cryogen such as LN2. The second is recovery of a biologically viable cell or cells after warming. Vitrification media, however, are often toxic to cells when the cells are warm. The time exposure of cells to vitrification media during dehydration and warming therefore (not “thawing” since ice is not formed) must be carefully controlled to avoid cellular injury.

Slow chilling speeds require high, relatively toxic concentrations of vitrification media, such as 60% w/w CPA concentration. At fast chilling speeds, lower, less toxic concentrations can be used. If chilling speeds of 10⁶° C./minute or greater were attainable, vitrification could be achieved with no cryoprotectants at all.

It is desirable to chill quickly; the faster the better. Directly plunging a biological specimen into LN2 achieves rapid chilling, but may expose biological specimens to contamination. Commercially available liquid nitrogen may contain bacterial and fungal species which are viable upon warming. Furthermore, it has been reported that vitrified cells can be infected by viral pathogens placed in the LN2.

The potential of infection has led to the development of closed cryocontainers where the biological specimen is placed in a container and sealed before chilling in LN2. The cryocontainer also serves as a storage device to isolate the biological specimen from pathogen-containing cryogen during long-term storage.

One of the limitations to achieving the fastest possible chilling speed with either direct plunge open cryocontainers or closed cryocontainers is that contact of the initially warm vitrification device with LN2 will result in the generation of an insulating nitrogen vapor coating around said device. The vapor barrier results from the boiling of the liquid nitrogen when it contacts the warm sample. This is known as the Leidenfrost effect. Nitrogen vapor is a thermal insulator which significantly diminishes chilling speed. If the Leidenfrost effect were reduced or eliminated, chilling speeds would be faster and less toxic vitrification media could be used which would potentially lead to better clinical outcomes.

There is a need therefore, for a method and apparatus to increase the cooling rate of biological specimens during vitrification by overcoming the Leidenfrost effect.

### SUMMARY OF THE INVENTION

The Summary of the Invention is provided as a guide to understanding the invention. It does not necessarily describe the most generic embodiment of the invention or all species of the invention disclosed herein.

### Improved Vitrification Chilling Apparatus and Method

The inventions described herein comprise chilling devices and methods that eliminate or reduce the Leidenfrost effect in vitrification. In the case where the chilling cryogen is a saturated liquid (e.g. LN2), high cryogen velocity is used to forcefully displace boiled-off vapors. In the case where the cryogen is a subcooled liquid chilled by a refrigerant, a simple means is described for controlling the temperature of the cryogen without freezing it.

In one embodiment of this invention, a saturated cryogen is held in a closed dewar whose exterior is exposed to room temperature. Ambient warming pressurizes the dewar and this pressure forces the saturated cryogen out of the dewar as a fluid jet. This jet is directed onto the vitrification device and forcefully displaces the evolved vapors to prevent formation of the Leidenfrost effect.

### BRIEF DESCRIPTION OF DRAWINGS

FIG. 1 illustrates two prior art vitrification devices. The first is a closed device and the second is an open carrier.

FIG. 2 illustrates an inventive closed vitrification device.

FIG. 3 illustrates the Leidenfrost effect on vitrification devices and means to mitigate it.

FIG. 4 schematically shows how pressure can be converted into cryogen velocity.

FIG. 5 illustrates two types of open contacting devices.

FIG. 6 illustrates a vessel-type contacting device.

FIG. 7 illustrates the features of an inventive dewar containing a saturated cryogen.

FIG. 8 illustrates the relationship between vapor pressure and temperature for nitrogen.

FIG. 9A illustrates the features of a closed contactor.

FIG. 9B illustrates a clamp for use with the closed contactor.

FIG. 10 illustrates how a contactor can be used to vitrify a biological specimen in an open cryocontainer.

FIG. 11 illustrates how a contactor with a converging/diverging nozzle can be used to vitrify a biological specimen in a closed cryocontainer.

FIG. 12 illustrates how a contactor that exhibits the vena contracta effect can be used to vitrify a biological specimen in a closed cryocontainer.

FIG. 13 illustrates a method to vitrify a specimen in a cryocontainer using a contactor and a self-pressurizing dewar of liquid nitrogen.

FIG. 14 illustrates the features of a dewar having two reservoirs. The first reservoir is for a saturated cryogen that pressurizes the dewar. The second is for a subcooled cryogen that will be used to vitrify a biological specimen.

### DETAILED DESCRIPTION

The following detailed description discloses various embodiments and features of the invention. These embodiments and features are meant to be exemplary and not limiting.

As used herein, except for temperature and unless specifically indicated otherwise, the term “about” means within +/−20% of a given value for a parameter. For temperature, “about” means +/−2° C. of a given value.

A variety of biological cells can be aseptically cryopreserved (vitrified) using the present invention. One category of cells is mammalian developmental cells such as sperm, oocytes, embryos, morulae, blastocysts, and other early embryonic cells. These cells may be cryopreserved during assisted reproduction procedures. Another category is stem cells that are used in regenerative therapies. The broadest category is any cell or group of cells that can be vitrified with the available chilling speeds of this invention.

### Vitrification Devices

Cryocontainers used as vitrification devices span a wide range of designs which can benefit from this invention. Exemplary cryocontainers are described below.

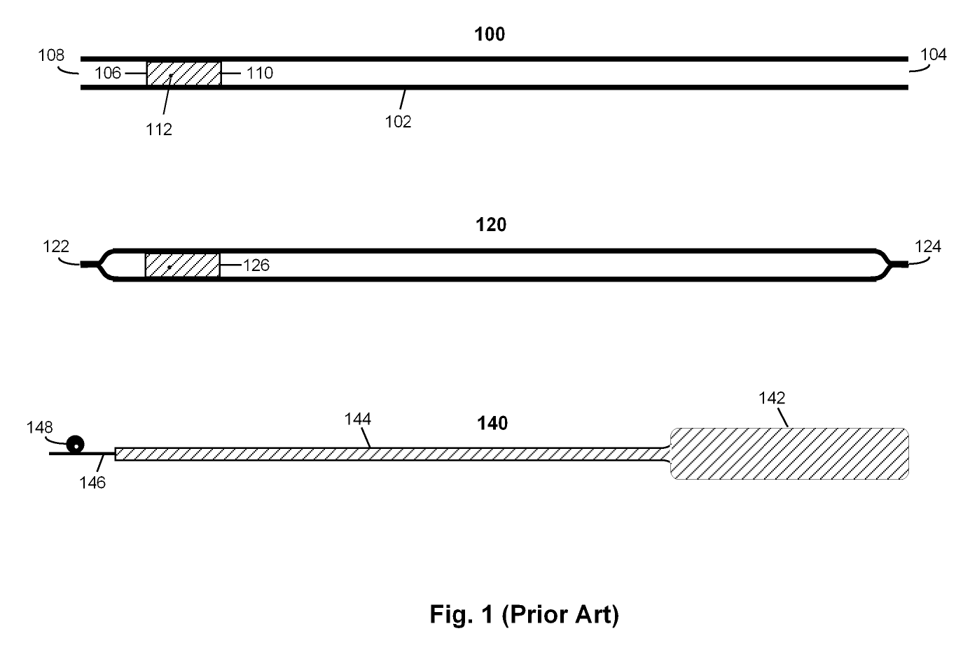

FIG. 1 shows longitudinal sections of generally tubular elements of an exemplary cryocontainer 100 comprising a cryocontainer tube 102. This device uses aspiration vacuum to draw a biological specimen into the cryocontainer tube. Both ends of the cryocontainer tube are initially open. A syringe (not shown) is attached to first opening 104 of the cryocontainer tube. The syringe creates a vacuum that draws biological specimen 106 into the second opening 108 of the cryocontainer tube. A biological specimen comprises vitrification media 110 and one or more cells 112. Referring to item 120, both ends of the cryocontainer tube are then heated and crimped to create aseptic seals 122 and 124. The cryocontainer containing biological specimen 126 is now prepared for chilling.

Item 140 in FIG. 1 illustrates an open carrier for vitrification. It comprises a handle 142 attached to a shaft 144. The shaft is attached to a loading cantilever 146. A biological specimen 148 is placed upon the cantilever.

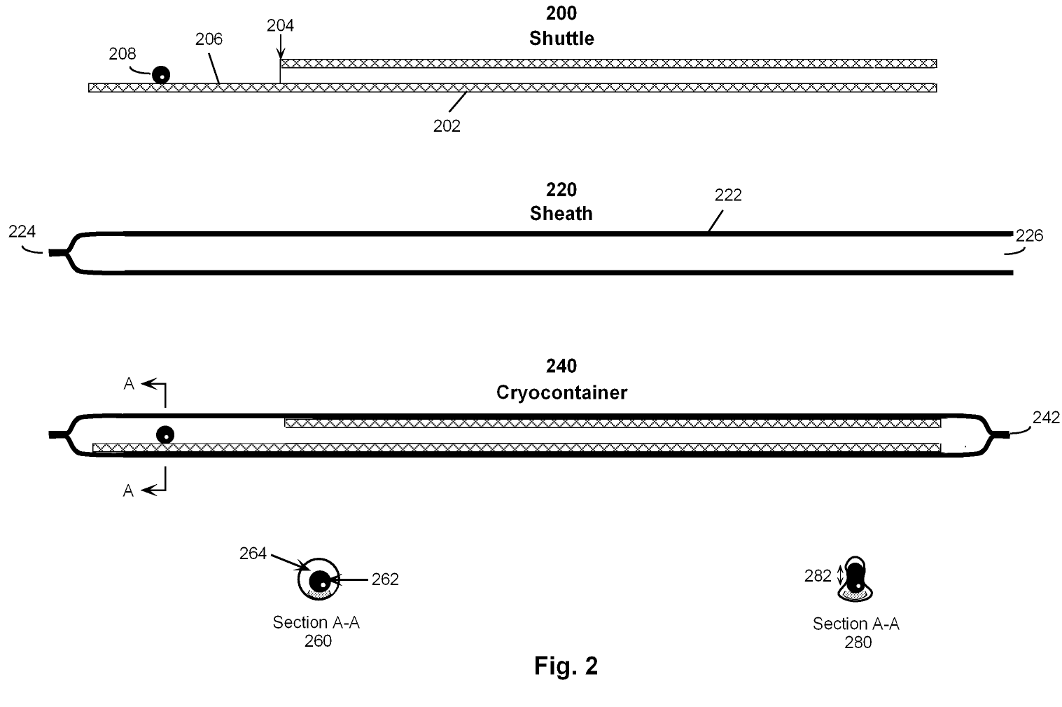

FIG. 2 illustrates longitudinal sections of generally tubular elements of an exemplary cryocontainer with deformable walls. Said walls may comprise a shape memory material. This cryocontainer is more fully described in copending US patent application “Shape-Shifting Vitrification Device”, Ser. No. 12/267,708. Said application and all continuations in part thereof, are incorporated herein by reference.

The cryocontainer comprises a shuttle 200 and sheath 220. The shuttle comprises a tube 202 with a notch 204 cut in the end to provide a channel 206. A biological specimen 208 is placed on the channel.

The sheath comprises a tubular body 222 with a first end 224 heat-sealed. A second end 226 is open. The portion of the sheath corresponding to the position of the biological specimen is deformable. Thus after the shuttle is loaded into the sheath, the deformable section may be crimped such that it touches the biological specimen. This increasing the heat transfer rate to said biological specimen. If the deformable wall is a shape memory material, it may return to its uncrimped shape when the cryocontainer is warmed. This facilitates removal of the biological specimen.

Item 240 shows the shuttle and sheath assembled and sealed 242, but prior to crimping.

Item 260 shows a cross section of the cryocontainer at section A-A immediately after loading and sealing. The biological specimen 262 is surrounded by air 264.

Item 280 shows the same cross section after crimping. A significant portion 282 of the deformable wall thus contacts the biological specimen. Thus the rate of heat transfer to the biological specimen will be increased during both cooling and warming.

### Heat Transfer

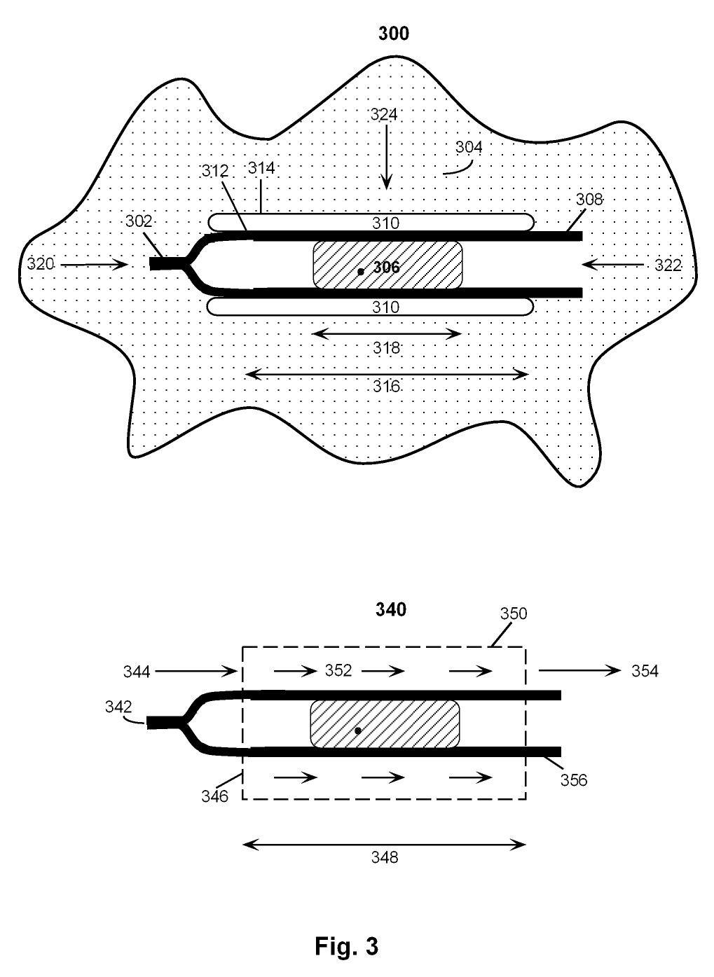

FIG. 3 illustrates how a cryocontainer reacts to being plunged by conventional means into a bath of LN2.

Item 300 shows the dynamics of the heat transfer in the portion of the cryocontainer 302 that contains the biological specimen 306. Plunging is performed into a quiescent pool 304 of LN2. The cryocontainer is within 20° C. of room temperature before the plunge. The contact of the LN2 with the relatively very hot cryocontainer surface 308 causes the LN2 to initially vaporize and form a vapor cloud 310 (stagnant gas) surrounding the cryocontainer. Subsequent heat transfer is from the cryocontainer's vapor coated surface 312 through the stagnant vapor to the opposite surface 314 having contact with the cryogen. The stagnant gas is nitrogen vapor and is a thermal insulator leading to low heat conduction rates. This is the so-called Leidenfrost effect. The chilling rate of the biological specimen is controlled by the rate that heat can be conducted across the stagnant gas layer.

If the cryocontainer's outside diameter is D, then we can estimate the heat transfer zone's length 316 to be wider than the footprint 318 of the biological specimen by about 2D. The rapid chilling methods described herein are preferably applied to the entire heat transfer zone.

According to the present invention, the Leidenfrost effect can be reduced if the cryogen is caused to flow at a high velocity over the cryocontainer. The cryogen velocity can be oriented in either direction along the principal axis of the cryocontainer, 320 and 322. Alternatively, the cryogen can be urged to flow transversely 324 across the cryocontainer or a suitable combination of transverse and longitudinal flow.

Item 340 illustrates the chilling of a vitrification device 342 using flowing cryogen 344 (“inbound stream”). Item 346 depicts a control volume within the flowing cryogen having a length equal to the heat transfer zone 348. We can assume, due to the jetting characteristics of the cryogen flow, that there is no heat or mass transfer across surface 350 of the control volume. The warm cryocontainer heats the flowing cryogen 352 within the control volume. Therefore, all the heat from the cryocontainer leaves the control volume in cryogen flow 354 (“outbound stream”). The cryogen flow across cryocontainer surface 356 induces convective heat transfer. This mode of heat transfer is superior to conduction through a stagnant gas layer. All the heat released by the cryocontainer leaves the control volume in the outbound stream.

This invention contemplates inbound streams having velocities that will renew the contents of the control volume hundreds of times during the time it takes to vitrify. Boiled-off vapors will be displaced out of the control volume by the momentum and shear forces arising from this velocity. A Reynolds number of at least 1,000 and more is suitable such that the flow is turbulent.

### Cryogen Velocity

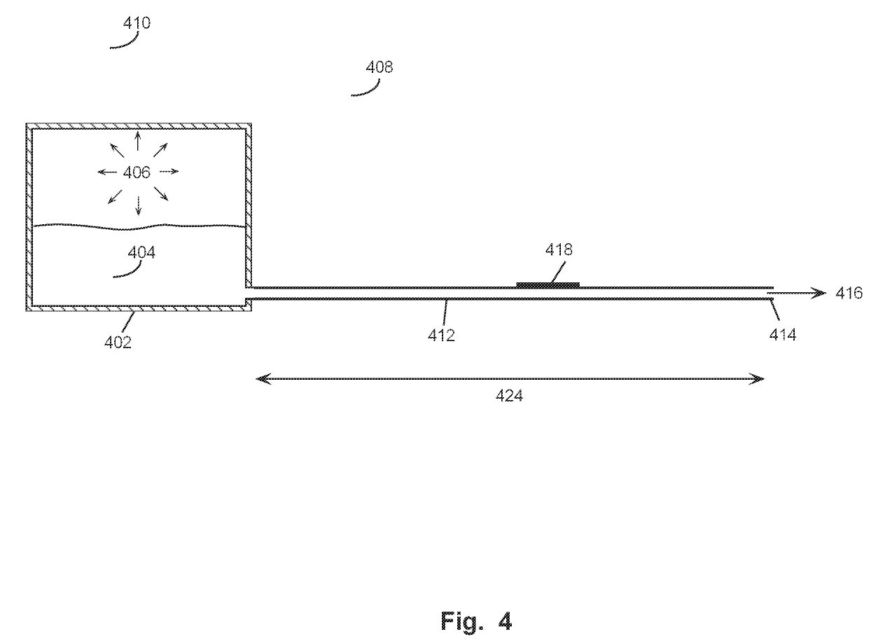

FIG. 4 illustrates a method for accelerating a cryogen to the required velocity. A container 402 with a cryogen 404 is at a pressure 406 above the pressure 408 of the surroundings 410. Leading from this container is a tube 412 with an exit 414 that empties into the surroundings. The pressure in the container urges the cryogen to flow through the tube and exit into the surroundings at a velocity 416. The fluid velocity at the exit is dependent on the pressure in the container and the frictional losses in the tube.

If the cryogen is a saturated liquid, such as LN2, some of it may flash into vapor as it passes through the tube. This can be minimized by insulating 418 the tube, keeping its length 424 short, minimizing frictional losses due to fluid motion, and using low thermal conductivity materials, such as plastics. The system is effective nonetheless even if a significant portion of the LN2 is in vapor form as it passes over the cryogenic container containing the biological sample.

Another method to accelerate a cryogen is to contact a high velocity gaseous non-condensing stream (e.g. helium) with the cryogen. This contact urges the gaseous stream and cryogen to form a moving entrainment stream.

The container with a saturated cryogen will create a high velocity of cryogen in exchange for the propensity to flash into vapor. The pressure of the container, therefore, can be adjusted through experimentation to give an optimum value for heat transfer for a given geometry of cryocontainer such that the velocity is high without undue flashing.

### Cryogen Contact Using an Open Contactor Apparatus

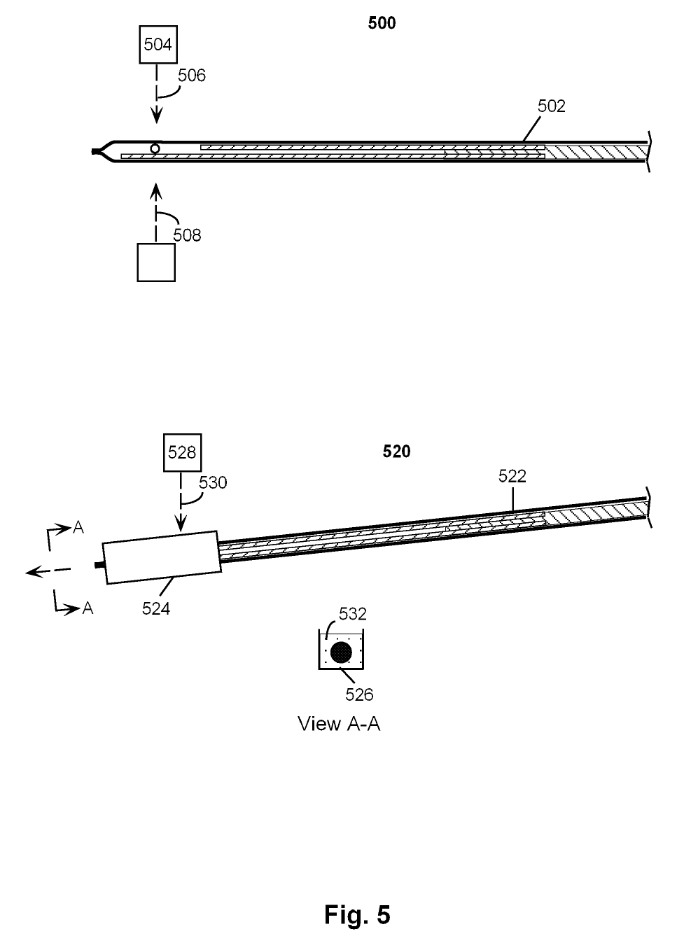

FIG. 5 illustrates open contactor apparati for contacting a high velocity cryogenic fluid with a cryocontainer.

Item 500 depicts a cryocontainer 502 in proximity to a cryogen velocity source 504. A cryogen velocity source is an apparatus that can urge a cryogen to flow in a desired direction. An example would be a pressurized container with an outlet pipe. The cryogen stream 506 is in the form of a jet with a preferred diameter of 5-10 times the heat transfer zone. It has a more preferred diameter of 1.5-3 times the heat transfer zone. The cryogen stream is directed onto the heat transfer zone to chill it and achieve vitrification of its contents. The cryogen stream can be a gas, subcooled liquid, saturated liquid or mixtures thereof. There may be a multitude of cryogen streams to insure complete coverage of the cryocontainer's periphery. An example would be two opposing streams 506 and 508. After vitrifying, the cryocontainer is placed in a bath of cryogen for long-term storage.

Item 520 depicts a cryocontainer 522 canted at an angle. Suitable angles are in the range of 2° to 45° from the horizontal. Surrounding the distal tip of the cryocontainer is a three-sided enclosure 524 that forms a channel 526 (View A-A). Suitable widths and heights of the channel are 1.5 to 10 times the diameter of the cryocontainer. Liquid cryogen from velocity source 528 forms a stream of cryogen 530 that impacts the cryocontainer at or near the heat transfer zone. The flow rate of the cryogen stream is sufficient to form a channel flow 532 (View A-A) that completely immerses the cryocontainer, chills it, and vitrifies its contents. After vitrifying, the cryocontainer is placed in a bath of cryogen for long-term storage.

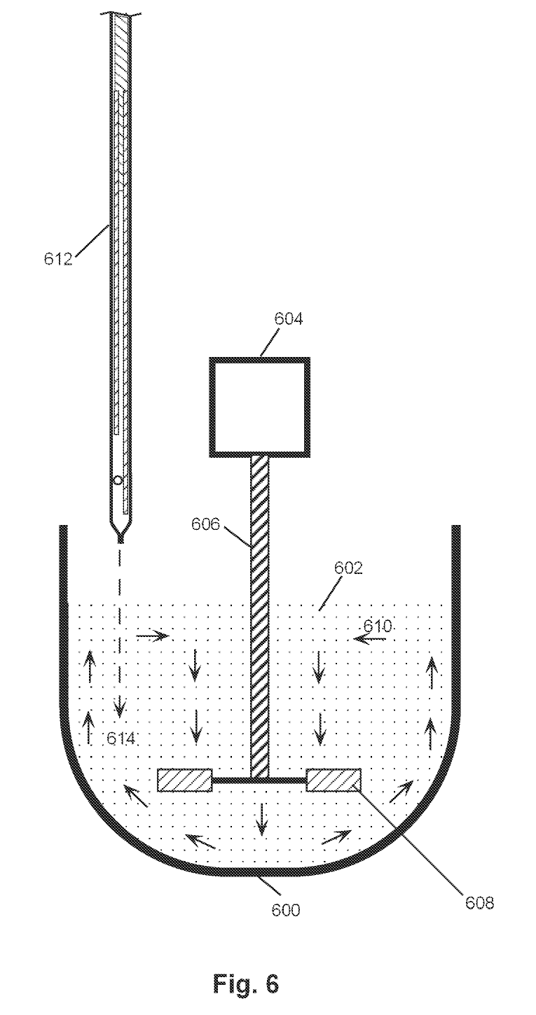

FIG. 6 depicts an open vessel 600 containing a liquid cryogen 602 that may be either a subcooled or saturated liquid. Attached to the vessel is an agitation system comprising a motor 604 that is connected to a shaft 606 that is connected to an agitator 608. The spinning agitator urges the contents of the vessel to flow in a manner depicted by arrows 610. The nature of the flow pattern formed may be different with an alternative agitation system. An external recirculation loop driven by a pump is a suitable alternative. A magnetic stirring system is also suitable. The key element is fluid velocity within the confines of the vessel. Before the plunge, the cryocontainer 612 is oriented with the heat transfer zone close to the cryogen's surface. The cryocontainer is then plunged into the cryogen to a depth 614 that immerses the heat transfer zone. Exposure to the cryogen vitrifies the contents of the cryocontainer. If the cryogen is a saturated liquid, the cryogen's velocity in the open vessel minimizes the Leidenfrost effect.

When vitrifying using an open cryocontainer to hold the biological specimen, the cryogen's velocity must be limited. This is to prevent displacing the biological specimen from the carrier and into the cryogen.

### Saturated Cryogen Dewar

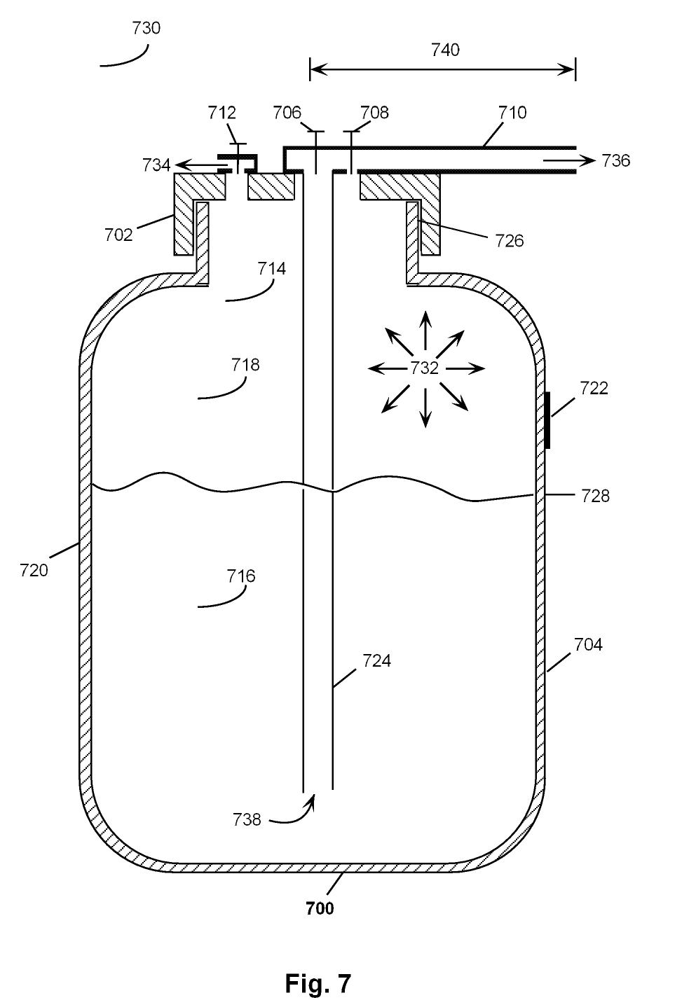

FIG. 7 illustrates the use of a pressurized container to generate a high velocity stream of cryogenic fluid. One way to pressurize a container of a saturated cryogen (such as LN2) is to place the cryogen in a sealed container, such as a closed dewar 700, and allow it to absorb ambient heat. The ambient heat then evaporates a portion of the cryogenic fluid thus raising the head space pressure.

The dewar comprises top assembly 702 and bottle 704. The top assembly comprises a release valve 706, a bleed valve 708, an outlet pipe 710, and a pressure relief valve 712. The bottle comprises a chamber 714 containing LN2 716 and head space 718 containing nitrogen vapor. The wall 720 of the bottle is a vacuum insulated double wall. The vacuum thermally insulates the contents of the bottle. The exterior of the bottle may be insulated 722 to protect the hands of the user from cold temperatures. Connected to the release valve is a dip tube 724 that extends into the bottle to draw out LN2. The top assembly is joined to the bottle by a threaded connection 726.

To use the dewar, the assemblies are first separated. A quantity of LN2 is poured into the bottle to form a level 728. The top assembly is then screwed onto the bottle.

The pressure relief valve has two settings: “purge” and “pressure”. In the purge setting, the pressure relief valve is opened and the head space pressure is the same as the ambient pressure 730. In the pressure setting, the pressure relief is set and the head space then pressurizes as ambient heat is absorbed by the LN2 and the LN2 vaporizes. The head space pressure 732 will rise until the set point of the pressure relief valve is reached. The relief valve will then open to release excess pressure 734 thus maintaining the LN2 at a constant pressure. Well insulated, hand-held dewars can maintain their setpoint pressure for a day by minimizing the amount of LN2 that boils off.

To dispense 736 LN2 from the dewar, the release valve 706 is opened to allow the head space pressure to urge the LN2 738 up the dip tube and out the outlet pipe.

An important characteristic of the flowing LN2 stream is the relative proportion of liquid and vapor nitrogen. It is desirable to have a high proportion of liquid nitrogen. To achieve this, the design of the dewar should utilize plastics or similar materials having low thermal conductivity and/or heat capacity for the dip pipe and outlet pipe so that a minimal amount of LN2 is boiled in chilling said pipes. The length 740 of the outlet pipe should also be kept at a minimum to limit this parasitic heating. The entire fluid pathway from the bottle to the surroundings should minimize frictional losses due to fluid motion. The total liquid content of the exit stream may be 80% by volume or greater.

To reduce parasitic heating, the bleed valve 708 can be left open between vitrification sessions. A small flow of cold gaseous nitrogen will then flow through and chill the outlet pipe. This flow also prevents backwash of ambient air into the outlet pipe. Air may contain moisture that can freeze on cold surfaces and potentially cause a blockage.

For some embodiments, it may be preferred to dispense gaseous nitrogen through the outlet pipe. This is easily achieved by utilizing a shorter dip pipe that draws from the head space rather than the LN2.

About 40 grams of LN2 might be consumed for the vitrification of a typical biological specimen held in a typical cryocontainer. Bleed nitrogen consumption to keep the exit pipe chilled and purged of ambient air might be 25 grams per hour. A typical laboratory vitrifying a plurality of specimens might require two devices so that one can be recharged with LN2 while the other is in use.

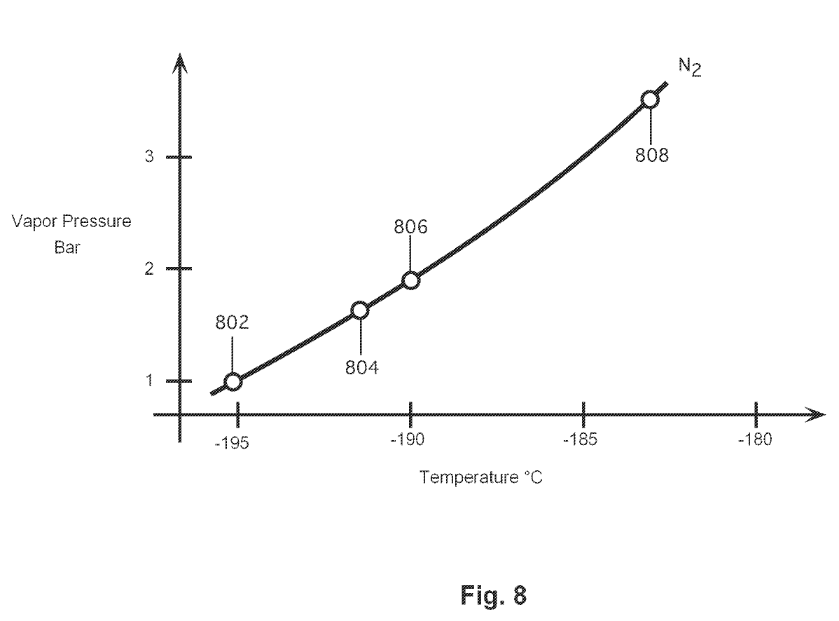

FIG. 8 illustrates the relationship between vapor pressure and temperature for LN2. If the pressure relief value of the cryogenic container of FIG. 7 is set to vent at 1 bar absolute, the corresponding temperature 802 is the normal boiling point of LN2, −196° C. If the pressure relief valve is set to 1.6 bar, the LN2 in the bottle will warm 804 to −192° C. Higher temperatures 806, 808 can be achieved with higher pressure settings. Higher temperatures might be required for applications where a second cryogen, such as propane or octafluropropane is held in the same dewar (see below). The higher temperature is necessary so that the second cryogen won't freeze.

### Cryogen Contact Using a Closed Contactor Apparatus

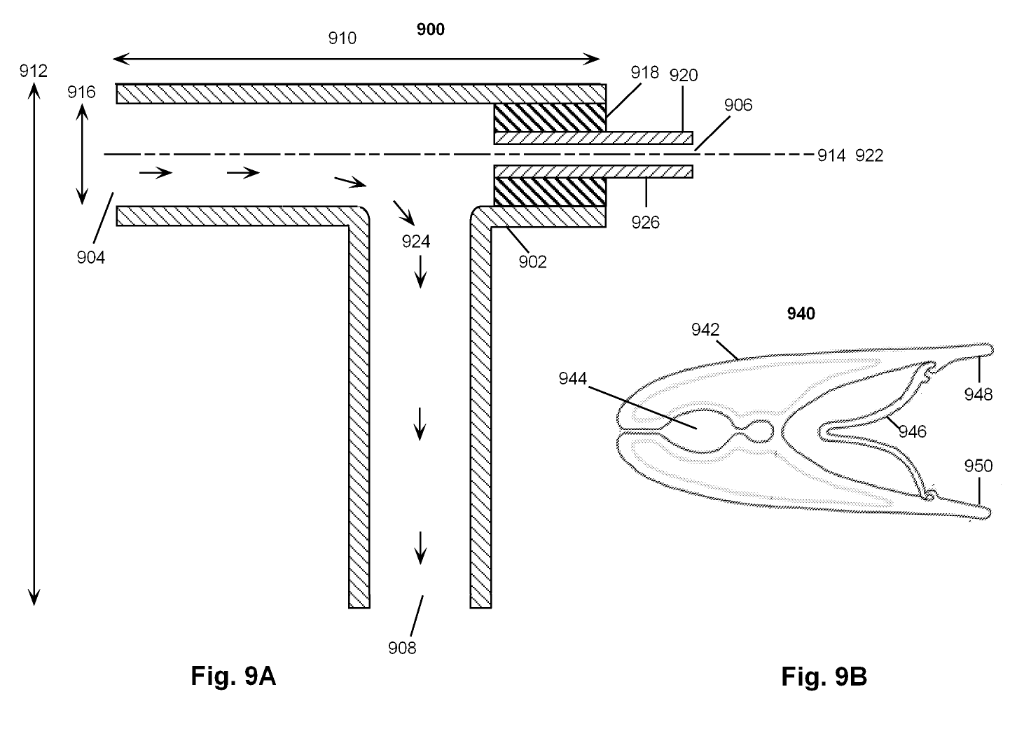

FIG. 9A illustrates a sectional view of a closed contactor apparatus 900 comprising a cylindrical body 902, a cryogen entrance 904, a cryocontainer opening 906 and an exhaust 908. The entrance is adapted to receive a flow of cryogenic fluid. The opening is adapted to admit said cryocontainer into said contactor. The body is adapted to direct said cryogenic fluid to said cryocontainer when said cryocontainer is loaded through said opening. The exhaust is adapted to direct said cryogenic fluid away from said cryocontainer when said cryocontainer is loaded through said opening.

The contactor's overall length 910 is about 6 cm. The length 912 of the exhaust is similarly about 6 cm. The cylindrical body comprises an axis 914. The ID 916 of the cylindrical body is about 1.7 cm. Suitable IDs of the cylindrical body can range from 1.5 to 10 times the diameter of the cryocontainer. Suitable diameters of cryocontainers can range from 100 microns to 2.5 mm. If a cryocontainer had a 2 mm diameter, for example, then a suitable ID of the cylindrical body would be 3 mm.

The cylindrical body further comprises a bushing 918 and a sleeve 920. The axis 922 of the sleeve may be coincident with the axis of the cylindrical body or it may be offset or at an angle thereto. The clearance between the ID of the sleeve and the cryocontainer should be kept to a minimum. However, the clearance should be sufficient to allow easy ingress and egress of the cryocontainer.

The cross section of the cylindrical body may be circular, square or other appropriate shape. If the cross section of the cylindrical body is different than a circle, then the “ID” of the body is the minimum distance across its cross section. Similarly, if the opening for the cryocontainer is noncircular, then the “ID” of the opening refers to the minimum distance across its cross section.

Materials to fabricate these parts should be suitable for cryogenic temperatures. Components that are in contact with the cryogen upstream of the heat transfer zone of the cryocontainer may have low thermal conductivity and low heat capacity. Plastics have these attributes. The sleeve needs to be flexible to allow clamping around the vitrification device. A suitable material for the sleeve is polytetrafluroethylene.

Referring to FIG. 9B, Clamp 940 comprises a body 942 having a jaw 944 that is held shut by spring 946. Moving handles 948 and 950 together opens up the jaw.

Referring back to FIG. 9A, the body of the contactor is attached to a source of flowing cryogen (not shown) at the cryogen entrance. Arrows 924 indicate the general flow of cryogen through the contactor. The diameter of the cryogen supply tube attached to the cylindrical body can be significantly larger than the diameter of said cylindrical body. It would be attached to the cylindrical body by a reducing union. This would minimize the pressure drop and hence flashing (if the cryogen is saturated) from the cryogenic supply reservoir to the closed contactor apparatus.

In order to vitrify, the end of a cryogenic container is inserted through the sleeve. The container is inserted until the heat transfer zone is located where it will be immersed within the general flow of the cryogen. Once in place, the open jaw of the clamp is placed over the sleeve at 926. The handles of the clamp are released which allows the jaw to squeeze the sleeve to engage the cryogenic container. The purpose of clamping the cryogenic container to the sleeve is to keep the cryocontainer from being pushed out of the sleeve when the cryogen flows. The clamped sleeve, however, does not need to be vapor or liquid tight. Other suitable clamps may also be used.

Once the cryogenic container is in place, the cryogen flow is initiated and the biological specimen is vitrified. The cryogen can be a gas, subcooled liquid, saturated liquid or a combination thereof. After vitrification, the clamp is released and the cryocontainer is placed in long term cryogenic storage.

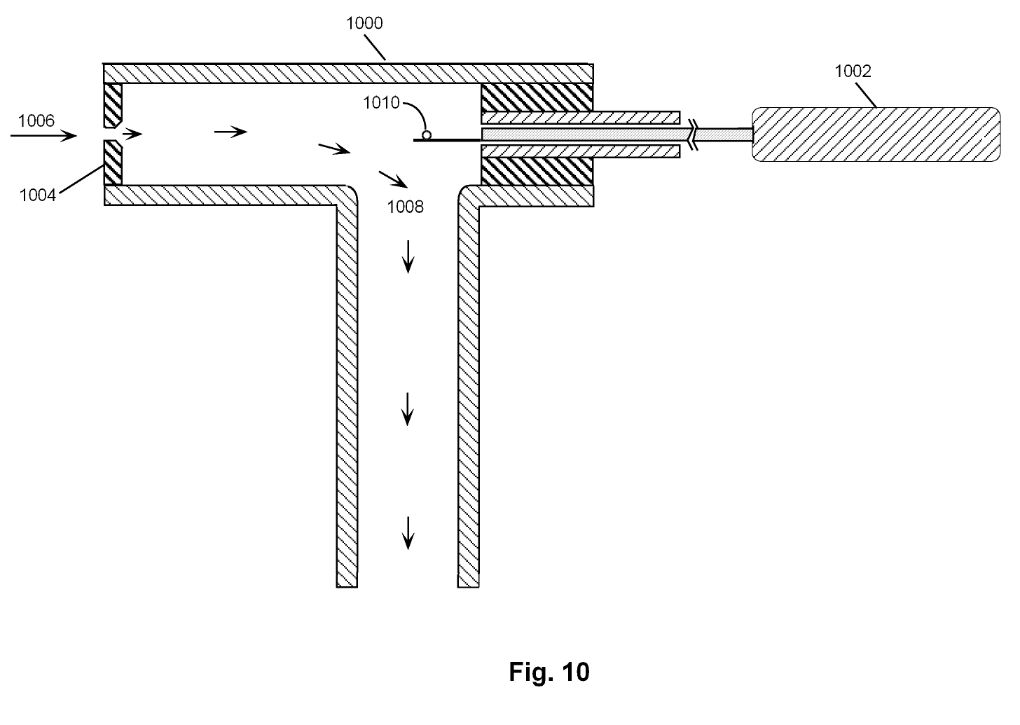

FIGS. 10-12 illustrate different embodiments of the closed contactor design. In all cases, the cryogen entrance is attached to a cryogen source that can dispense either a gas, subcooled liquid, or a saturated liquid. Also, different vitrification devices can be engaged as described above in the illustrations of the embodiments.

FIG. 10 illustrates a contactor 1000 loaded with an open vitrification device 1002. Due to the delicate nature of an exposed biological specimen 1010, the contactor may also be comprised of a restricting orifice 1004 at the cryogen entrance. It may also be preferred to have the cryogen source to be a gas. Cryogen dispensed 1006 traverses the contactor as shown by the arrows 1008. The flow of cryogen contacts the open carrier and vitrifies the biological specimen.

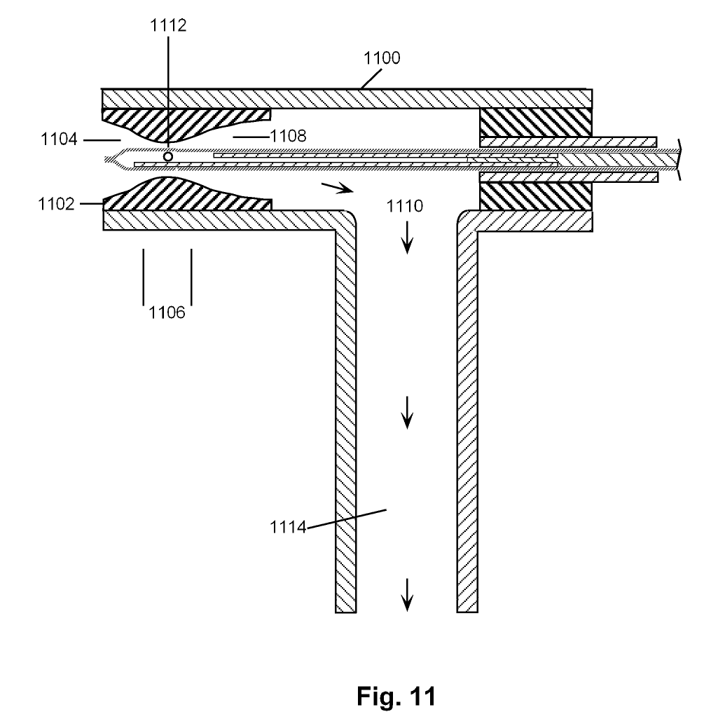

FIG. 11 illustrates a contactor 1100 loaded with a closed vitrification cryocontainer. A converging/diverging nozzle 1102 is designed to shape 1104 the flow of the incoming cryogen to a very narrow annular stream in the proximity 1106 of the heat transfer zone. Smooth transitions 1108 are needed on both sides of the heat transfer zone to preserve the pressure for conversion to velocity. The flow of cryogen 1110 vitrifies the biological specimen 1112. The gap between the vitrification cryocontainer and the nozzle may be 0.2 to 1.5 mm. The upstream pressure may be 0.2 to 3 bar above ambient. The pressure 1114 in the exhaust is ambient. Therefore, virtually all of the pressure drop occurs in the nozzle. Cryogen velocity in the heat transfer zone may be 0.5 to 25 meters per second (m/s). This velocity is sufficient to prevent the formation of the Leidenfrost effect.

The axis of the contactor body may be about coincident with the axis of the opening for the cryocontainer such that when the cryocontainer inserted through said opening, the heat transfer zone can be located in the throat of the converging diverging nozzle.

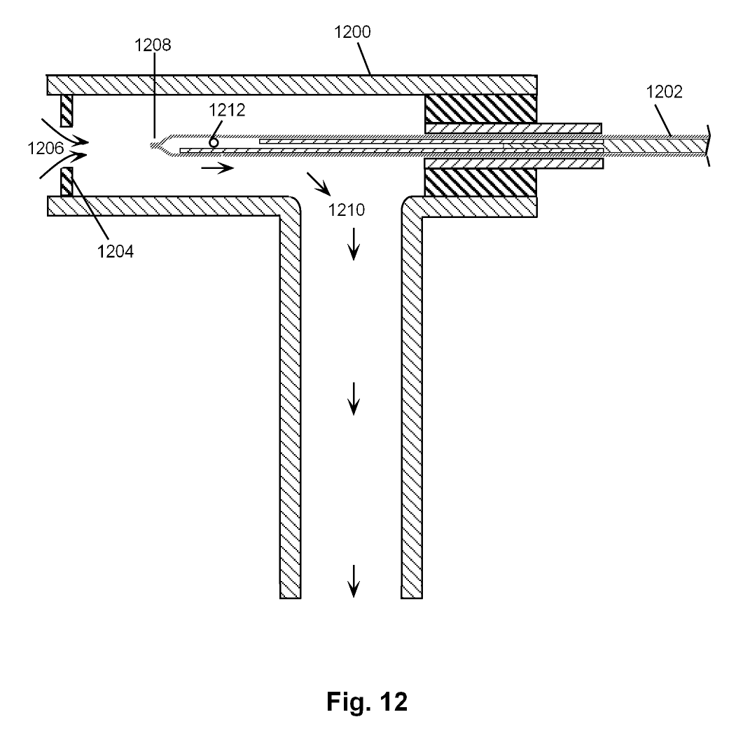

FIG. 12 shows a contactor 1200 loaded with a closed vitrification cryocontainer 1202. A flow orifice 1204 is designed to shape 1206 the flow of the incoming cryogen to a downstream vena contracts 1208 in the proximity of the heat transfer zone. The flow of cryogen 1210 vitrifies the biological specimen 1212.

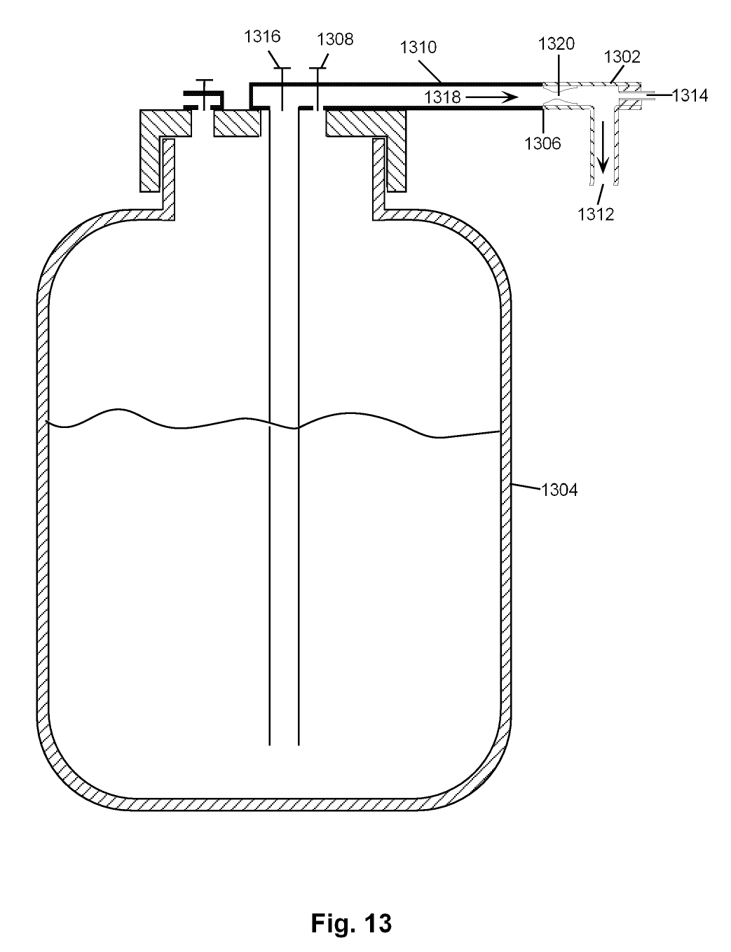

FIG. 13 illustrates a contactor 1302 attached 1306 to a dewar 1304 containing LN2. Prior to vitrification, cold gaseous nitrogen gas from a bleed valve 1308 travels through an outlet pipe 1310. This chills the contactor to limit parasitic heating. This flow also prevents ambient air that may contain moisture, from entering the contactor at the exhaust 1312 or cryocontainer opening 1314.

During vitrification, a cryocontainer is placed through the opening and the LN2 is turned on by opening release valve 1316. The LN2 velocity 1318 in the outlet tube is relatively slow as compared to its velocity 1320 in the heat transfer zone. This is highly desirable as it reduces the pressure drop from the dewar to the contactor. The pressure in the head space of the dewar is set high enough such that the velocity of the LN2 in the heat transfer zone is at least 0.5 m/s.

### Two Chamber Dewar

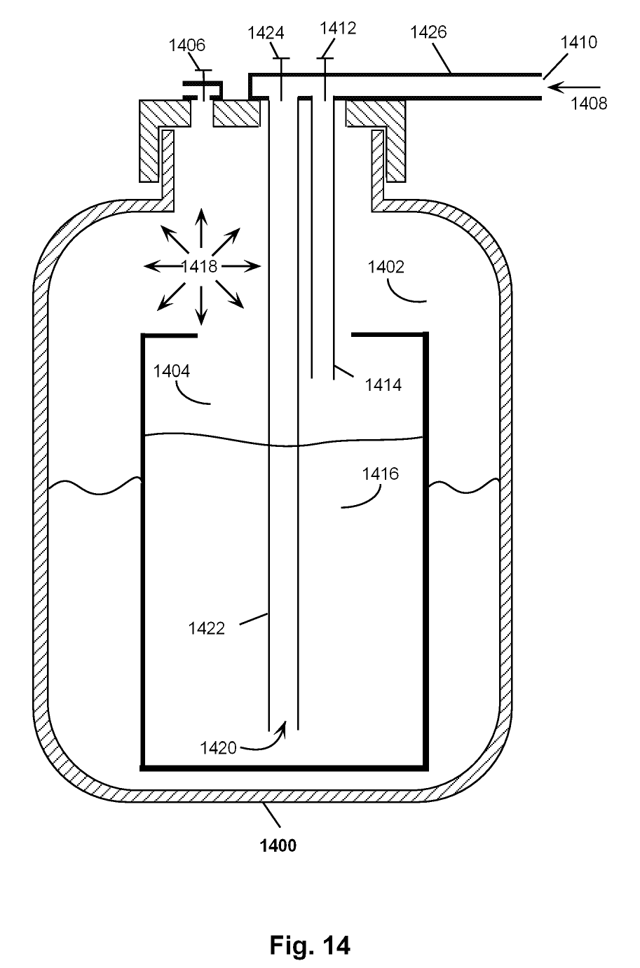

FIG. 14 illustrates how a saturated cryogen dewar can be used to dispense a subcooled cryogen. An exemplary subcooled cryogen is propane which has a boiling point of −42° C. and a freezing point of −188° C. If propane is cooled to just above its freezing point, it can be used as a flowing cryogen with minimal potential for Leidenfrost effect.

Item 1400 is a double-chambered dewar wherein the first chamber 1402 serves as a reservoir for LN2. The second chamber 1404 serves as a reservoir for subcooled propane. The headspaces of the two reservoirs are in physical communication with each other such that the pressures therein are about equal. The first and second reservoirs are also in thermal communication with each other such that their temperatures are about equal.

The pressure relief valve 1406 has a set point that raises the head space pressure such that the LN2 temperature is just above the freezing point of propane. Two bar absolute is a suitable pressure (item 806, FIG. 8).

To load the second chamber, a source of propane gas 1408 is attached at the outlet 1410 of the dewar. A bleed valve 1412 is opened which allows the propane gas to enter the second chamber through a vapor dip pipe 1414. Heavier gaseous propane will displace the gaseous nitrogen and condense into a liquid 1416 and accumulate in the second chamber. An external scale can be used to control the loading of the propane. When the filling is complete, the propane source is disconnected.

The vapor dip pipe now communicates with gaseous nitrogen in the head space so the bleed valve reverts its function back to providing bleed nitrogen gas.

To dispense liquid propane, head space pressure 1418 urges the liquid propane 1420 to move through a dip pipe 1422 and hence though a release valve 1424 and into the outlet tube 1426. A contactor can be attached to the dewar at the outlet to enable vitrification with the liquid propane.

Propane is a flammable substance. A suitable non-flammable cryogen is octafluropropane (R218) having a boiling point of −37° C. and a freezing point of −183° C. Octafluropropane requires a head space pressure of 3.5 bar absolute to prevent freezing of the cryogen (item 808, FIG. 8).

### CONCLUSION

While the disclosure has been described with reference to one or more different exemplary embodiments, it will be understood by those skilled in the art that various changes may be made and equivalents may be substituted for elements thereof without departing from the scope of the disclosure. In addition, many modifications may be made to adapt to a particular situation without departing from the essential scope or teachings thereof. Therefore, it is intended that the disclosure not be limited to the particular embodiment disclosed as the best mode contemplated for carrying out this invention.

I claim:

1. A contactor for the rapid chilling of a biological specimen held in a cryocontainer, said contactor comprising:
a. an entrance, said entrance adapted to receive a flow of a first cryogenic fluid;
b. an opening, said opening adapted to admit said cryocontainer into said contactor; and
c. a body, said body adapted to direct said cryogenic fluid to said cryocontainer when said cryocontainer is loaded through said opening;
d. an exhaust, said exhaust adapted to direct said first cryogenic fluid away from said cryocontainer when said cryocontainer is loaded through said opening
wherein the ID of said body is at least 1.5 times the ID of said opening.

2. The contactor of claim 1 wherein said contactor is a closed contactor and wherein said opening comprises a sleeve, said sleeve having an ID in the range of 100 microns to 3 mm and wherein the ID of said body is not more than 10 times the ID of said sleeve.

3. The contactor of claim 1 which further comprises a source of said first cryogenic fluid, wherein said source of said first cryogenic fluid is a sealed dewar comprising a pressure relief valve, said pressure relief valve being adapted to maintain a pressure in the head space of said dewar such that said first cryogenic fluid can be accelerated to a velocity of at least 0.5 m/s when it passes through said body and wherein said source of said first cryogenic fluid is connected to said entrance.

4. The contactor of claim 3 wherein said dewar has a first reservoir for said first cryogenic fluid and a second reservoir for a second cryogenic fluid, and wherein the head spaces of said first reservoir and said second reservoir are in communication with each other such that the pressures in said head spaces are about equal.

5. The contactor of claim 4 wherein said first cryogenic fluid is liquid propane and said second cryogenic fluid is liquid nitrogen and wherein said pressure relief valve is set to a pressure such that the temperature of said liquid nitrogen is above the freezing point of said propane.

6. The contactor of claim 3 which further comprises a valve in communication between said head space of said dewar and said body such that the gases in said head space may be bled into said body.

7. The contactor of claim 2 wherein said sleeve comprises a flexible tube that is adapted to form a seal with said cryocontainer when said tube is clamped.

8. The contactor of claim 1 wherein said body comprises a converging/diverging nozzle.

9. The contactor of claim 1 wherein said body comprises an orifice dimensioned such that said first cryogenic fluid can form a vena contracta after passing therethrough.

10. The contactor of claim 1 wherein said body comprises a plastic suitable for cryogenic service.

11. The contactor of claim 7 wherein said sleeve comprises polytetrafluroethylene.

12. The contactor of claim 1 wherein the cross section of said body is square.

13. A closed contactor apparatus for the rapid chilling of a biological specimen, said apparatus comprising:
a. an entrance;
b. a cylindrical body, said cylindrical body comprising a converging/diverging nozzle;
c. an exhaust; and
d. an opening for admitting said cryocontainer;
wherein the axis of said cylindrical body is about coincident with the axis of said opening such that a cryocontainer inserted through said opening would pass through the throat of said nozzle.

14. The apparatus of claim 13 which further comprises a dewar, said dewar being in communication with said entrance such that a cryogenic fluid may flow from said dewar through said cylindrical body.

15. A closed dewar, said dewar comprising:
a. a first reservoir for containing a first cryogenic fluid;
b. a second reservoir for containing a second cryogenic fluid; and
c. a pressure relief value set to maintain a pressure in the head space of said first reservoir above ambient pressure;
wherein the head space of said first reservoir and the head space of said second reservoir are in physical communication with each other such that the pressure in the head space of said first reservoir is about equal to the pressure in the head space of said second reservoir and wherein said first reservoir is in thermal communication with said second reservoir such that the temperature of said first reservoir is about the same as the temperature of said second reservoir.
